(19) United States
(12) Patent Application Publication
(10) Pub. No.: US 2014/0163408 A1
(43) Pub. Date: Jun. 12, 2014

(54) System for analyzing mental and behavioral correlations

(71) Applicant: Ideal Innovations Incorporated, Arlington, VA (US)

(72) Inventor: Robert Kocher, McLean, VA (US)

(21) Appl. No.: 14/102,381

(22) Filed: Dec. 10, 2013

Related U.S. Application Data
(60) Provisional application No. 61/735,216, filed on Dec. 10, 2012.

(51) Int. Cl. A61B 5/0478; A61B 5/00

(52) U.S. Cl. CPC A61B 5/0478; A61B 5/6803
USPC 600/544

(57) ABSTRACT

A system and that detects the brain activity of one or more subjects using EEG sensor devices, analyzes the brain activity to detect information indicating certain physical conditions or future behavior of the one or more subjects, and uses the information indicating physical conditions or future behaviors to make decisions concerning the one or more subjects or the organization relating to them.

### CROSS-REFERENCE TO RELATED APPLICATION

This application claims the benefit of U.S. provisional patent application Ser. No. 61/735,216.

### BACKGROUND

The subject matter of this specification relates to the field of electroencephalography (“EEG”) and using information from EEGs to aid decision-making. The fluctuations of electrical potential in the brain, “brainwaves,” are unique to each individual, but there are patterns that provide specific indications linked to various stimuli. The electric potential measured by an EEG is the direct result of a specific sensory, cognitive, or motor event called an event-related potential (“ERP”). (Responses to less complex stimuli, such as the processing of a basic physical stimulus, are called evoked potentials (“EV”). For the purposes of this specification, the phrases “patterns of brain activity”, “brainwaves”, “brain patterns” and the like are intended to include ERPs and EVs.) By studying the ERPs that occur when a subject is experiencing a set of circumstances, it can be determined that certain ERPs or patterns of ERPs are markers for expectations, attentions, or other higher level mental phenomena of the subject during those circumstances. More generally, baseline EEG studies over various circumstances can be used to construct “normal” patterns of electrical activity for a subject. These normal patterns can then be used to determine if abnormal stimuli are present.

The problem of deciding the most optimal functions for individuals or sets of individuals within an organization has historically been limited to analyzing the available, objective characteristics of each individual or group. For example:
(i) A coach of a football team may choose the next play for his team based on the fact that his wide receiver has a significant height advantage over the opposing defensive back.
(ii) A CEO may choose the next marketing director based on sales history and professional education.
(iii) A police commander may choose investigative teams based on past arrest records.

However, it is often apparent that even the best objectively reasoned decisions fail because of unknown subjective facts about the individuals involved. For example:
(i) The quarterback throwing the ball to the wide receiver had a concussion from the previous play.
(ii) The next marketing director planned to move to a competitor in three months and has a worsening drug addiction.
(iii) A member of one of the investigative teams recently found out his wife wants a divorce.

Furthermore, too often a decision-maker is left wondering: “is his head in the game?”; “is something is bothering him?”; “is she up to doing it?”; “are last night's activities affecting him today?”; “is he ready for this?” Therefore it would be advantageous to develop the capability to incorporate subjective private facts of personnel into decision-making about those personnel.

### SUMMARY

The above decision-making problem is solved by a system and method embodying the principles of the subject matter of this specification that detects the brain activity of one or more subjects, analyzes the brain activity to detect information indicating certain physical conditions or future behavior of the one or more subjects, and uses the information indicating physical conditions or future behaviors to make decisions concerning the one or more subjects or the organization relating to them.

### DESCRIPTION OF THE SEVERAL VIEWS OF THE DRAWINGS

FIG. 1 depicts the primary components of the system embodying the principles of the subject matter of this specification.

FIG. 2 depicts an embodiment of the subject matter of this specification applying to a football player playing in a game.

FIG. 3 depicts a method performed when detecting the electrical activity in a subject's brain.

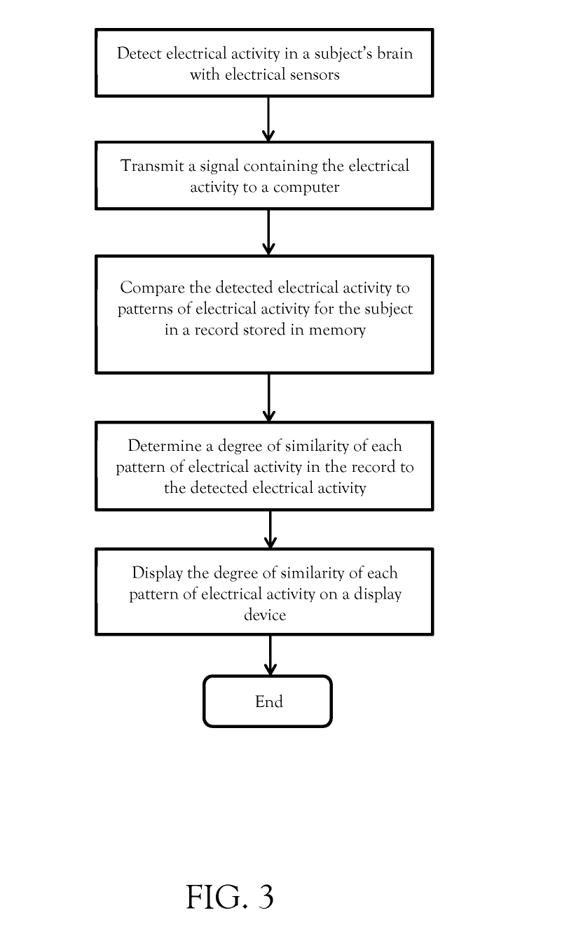

FIG. 4 depicts a method performed when evaluating the probability of a tactic.

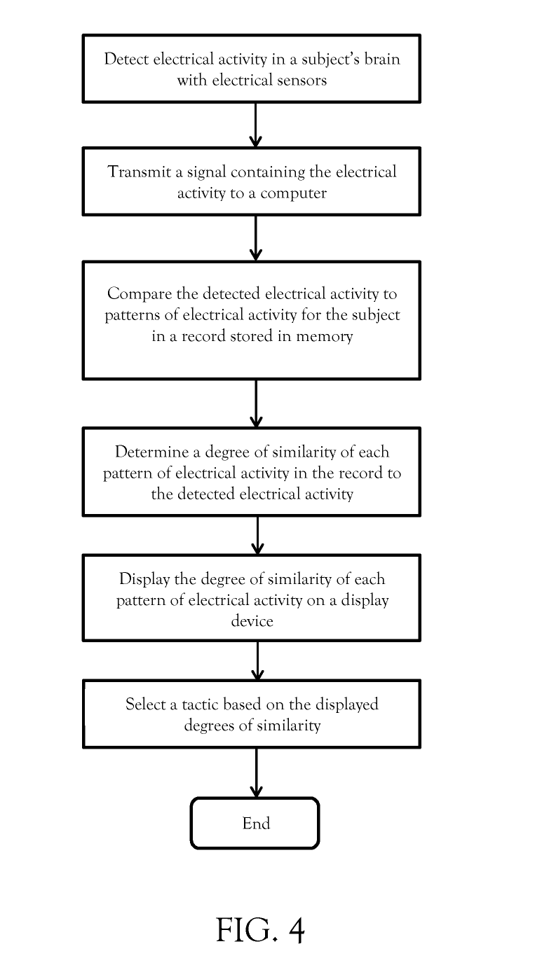

FIG. 5 depicts a method performed when creating a record of brain activity patterns of a subject.

### DETAILED DESCRIPTION

The subject matter of this specification collects performance circumstances and brainwave data of a subject over a period of time and develops a specific linkage between the subject's brainwaves and performance by looking at brainwave graphs and overlaying performance graphs during the same time period. A relationship algorithm looks at historical brainwave data and predicts positive or negative performance. A feedback loop can be added to improve the relationship algorithm and thereby improve predictive performance. In other words, when a high performer was performing at a high level of performance, what was his brain activity? The converse is also valuable—when a high performer was performing poorly, what was his brain activity?

EEG can be quantified in various ways by applying a Fourier transformation, including by amplitude, power, frequency, and rhythmicity in order to generate numerical values, ratios, or percentages; graphically display arrays or trends; and set thresholds. Many quantitative EEG measures can be used to quantify slowing or attenuation of faster frequencies in the EEG. These include the calculation of power within different frequency bands (i.e., delta, theta, alpha, and beta); ratios or percentages of power in specific frequency bands; and spectral edge frequencies (based on the frequency under which x % of the EEG resides). These discrete values can then be compared between different regions, such as hemispheres, or between electrode-pair channels. Time-compressed spectral arrays (“Spectrograms”) incorporate both power and frequency spectrum data and can be represented using color to show power at different frequencies. Additional measures include amplitude integrated EEG, which continuously monitors comatose patients by average ranges of peak-to-peak amplitudes displayed using a logarithmic scale, and the commercial Bispectral Index. Other nonparametric methods exist beyond Fourier transformation, including interval or period analysis and alternative transformation techniques. Parametric, mimetic, and spatiotemporal analyses are also available using a variety of computational methods and waveform analysis based on machine learning approaches trained on ICU EEG recordings. Basic measures of total power can be quantified and compared to performance characteristics to identify correlations that can be used to predict the reoccurrence of those performance characteristics.

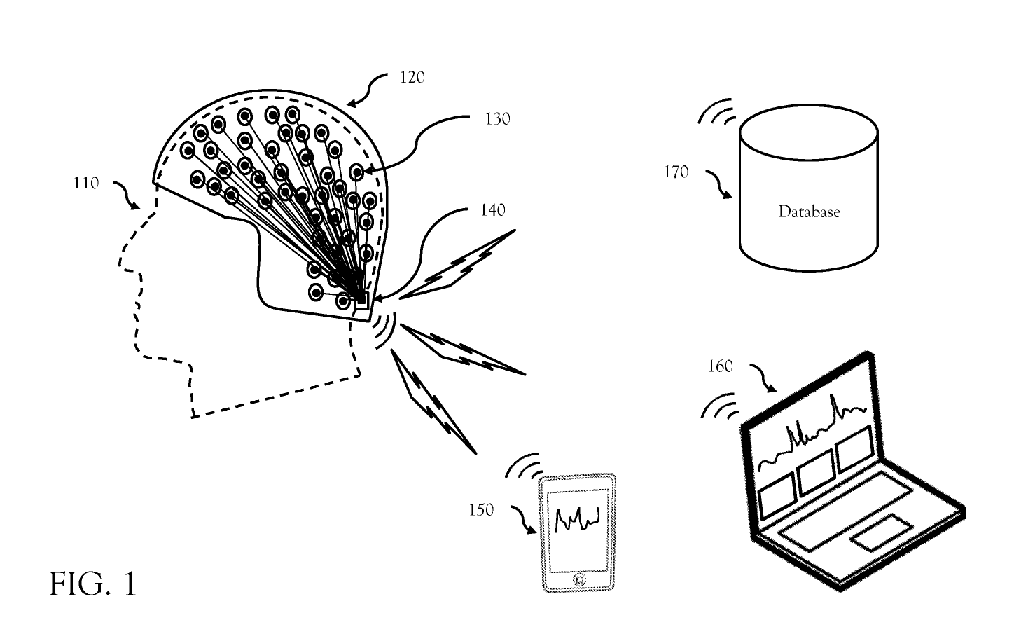

FIG. 1 depicts a system that comprises a basic embodiment of the components of subject matter of this specification. In FIG. 1, subject 110 is shown wearing a sensor device 120 having EEG electrodes 130 incorporated into a helmet. A range of suitable EEG devices are well known in the art and include devices such as that disclosed in figure one of European Patent No. EP2211712 and figure three of U.S. Pat. No. 1,238,9722 (each of which is hereby incorporated by reference), those offered by the OpenEEG project, Emotiv BCI, or Mattel Mind Flex, high density electrode devices such as Vision Lab's EEG harness, and high capacitive devices such as that developed by the Technische Universitat Carolo-Wilhelmina. In FIG. 1 the electrodes are incorporated into a sports helmet such as a football helmet that would be used on an athletic field. However, alternative embodiments the sensor devices may stand alone or in combination with other elements for diagnostic purposes.

The electrodes depicted in FIG. 1 are connected to a wireless communication component 140 incorporated into the helmet. This component may be any of the wireless radio devices known in the art that work on standards for Wi-Fi, cellular data service, mobile satellite communications, or similar wireless communication standards. Examples of such components include those manufactured by Cisco and Netgear, as well as the wireless functionality provided common mobile telephones. The wireless communication component 140 is show in FIG. 1 operatively connected to the other components of the embodiment by means of a wireless network. However, it should be noted that wireless communication is not necessary in all embodiments and hard wire connections are also possible such as by USB cable.

FIG. 1 depicts the mobile device 150, computer 160, and database 170. The mobile device may be any of those mobile devices commonly known in the art such as mobile telephones, smart phones, tablets, and minitablets. The computer may be any of those commonly known in the art having a processor, memory, and an input and display functionality. Database 170 may be a server or set of servers. The mobile device, computer, and database each incorporate wireless radios in a similar manner as the helmet, however wireless communication is not necessary in all embodiments and hard wire connections are also possible. Moreover, only the sensor device 110, and the computer 160 or mobile devices 150 are necessary for operation of the subject matter of this specification. Any software may run and be stored on the database and accessed remotely from the mobile device or computer; or software may run and store information entirely on the computer and/or mobile device without the database.

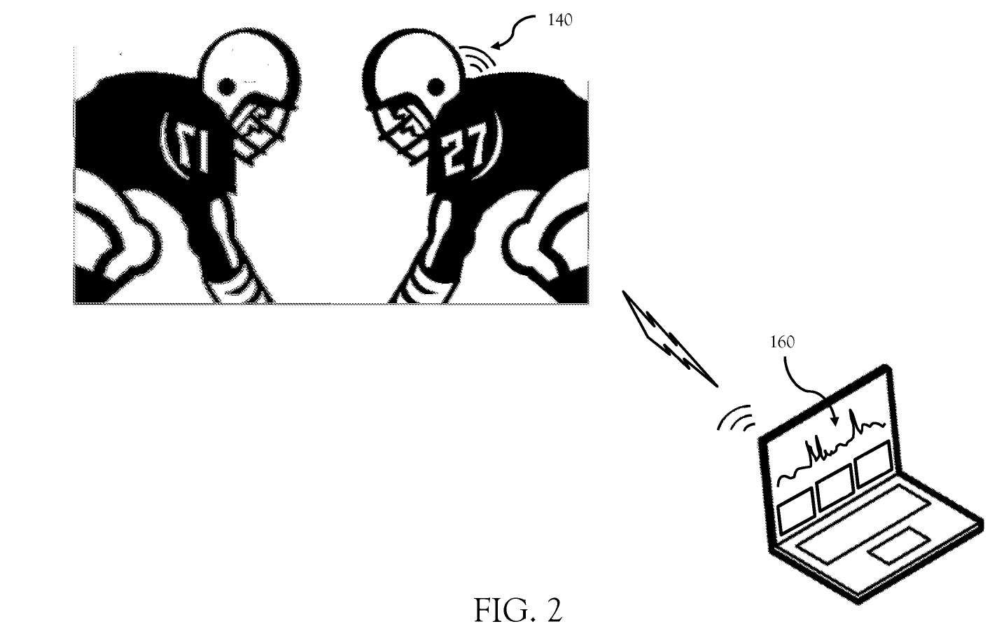

FIG. 2 depicts another view of the embodiment depicted in FIG. 1. In FIG. 2 a football player is shown playing in a game. The sensor device is incorporated into the player's helmet. Wireless communication device 140 connects the sensor device to computer 160 where brain patterns emitting from the player's brain are measured and displayed on the display device of the computer. With this information a person on the sidelines, such as a coach, can gain insight into the player's intention or level of attention, as well as the other physical capabilities of the player. The coach does not have to wait and observe failure. Instead he can proactively act to avoid a predicted failure. A coach would not have to wait for a quarterback to throw interceptions, the coach would see ahead of time that the quarterback is prone to errors by low performance indicators. The coach would not have the quarterback throw passes unless the quarterback's performance indications improved. The coach can also avoid mistakes by putting the backup quarterback in. The subject matter of this specification allows managers to “get inside a person's head” before and after a play. The results would be to maximize actions for which the team is most receptive and avoid actions for which the team is not it sync.

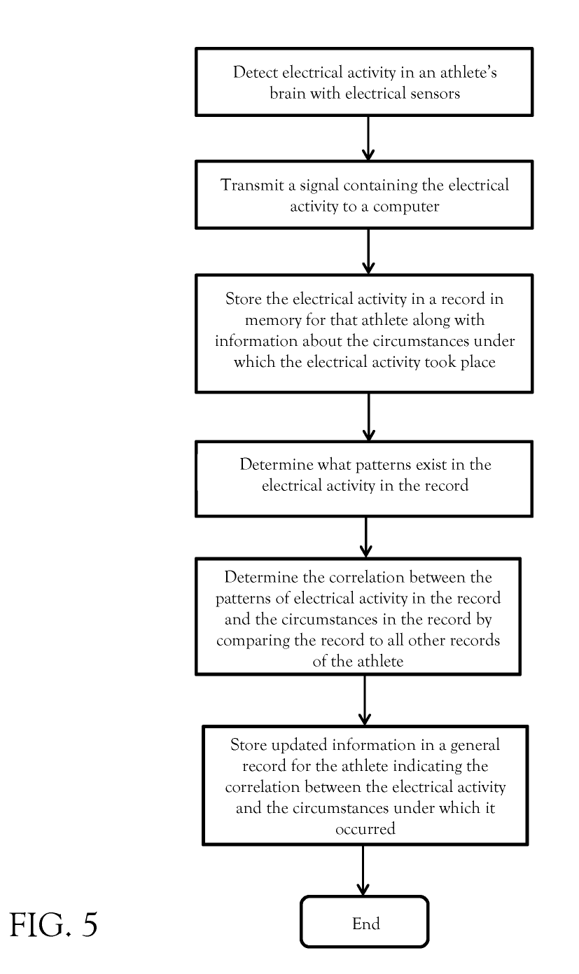

In the depiction shown in FIG. 2 the coach would first have to have acquired or created a database of relevant patterns of brain activity. FIG. 5 depicts the method for doing so. In accordance with the method depicted in FIG. 5, one may build an electronic record stored in memory of a computer of the relevant patterns of brain activity for a given individual. First an individual is properly outfitted with a sensor device as described above. The sensor device is operatively connected to a computer or mobile device having software installed thereon for controlling the sensor device. EEG control software is well known in the art and is manufactured by many of the same companies listed in the description of FIG. 1.

Second, the subject would be placed into a set of circumstances that are of interest. In one example, the subject could be a wide receiver running a particular route, on a particular surface, against particular type of pass defense. Simultaneously, the subject's observed performance data is collected through other means, such as a video camera, heart monitors, or observer input. As the subject progresses through the selected circumstance (runs the pass route) the entire set of data is captured and stored in memory. The process is repeated as many times as is necessary to acquire a statistically significant number of trials necessary in order to draw inferences from the sensor device data. Through statistical methods well known in the art, irrelevant artifacts may be excluded from the sensor device data and relevant patterns can be identified. These patterns in any given trial are then compared to the circumstantial data for that trial in order to find correlations between events of interest and patterns of brain activity. For instance it may be the case that the receiver drops significantly more passes when there is a defender he thinks is a hard hitter on the other side. When the receiver lines up, the sensor device may detect a particular pattern of brain activity during those plays. In another set of circumstances, the receiver may often miss the quarterback's signal when a play is changed at the line of scrimmage. But when he gets the signal his brain patterns exhibit a noticeably different pattern indicating recognition.

Once sufficient data has been collected and relationships are found, performance relationship data file is generated to serve as the predicting basis for that particular subject. In the football example a coach may monitor the signal of the receiver before he calls the play. If the receiver is indicating he is aware of a hard hitter he may not call a play for him in that situation. Or, if the quarterback changes the play and the coach recognizes the brain pattern indicating that the receiver did not get the change, the coach can contact the quarterback or call a time out.

The database for a given subject can continually be added to and refined as more circumstances are tested and correlations and patterns are strengthened or weakened. What might initially start out as a basic subject-focused set of data regarding a subject's patterns on certain plays or field conditions, may expand into indicators of that subject's success levels with each offensive unit under a variety of game circumstances as well as each other team member's similar records with respect to that subject. Even the opponent's players may be monitored to take advantage of indicators that predict weaknesses.

It can be readily seen that included in the circumstances under which a subject is evaluated, may be the presence or absence of other participating individuals. Thus the effects of other individuals on a subject's performance may be measured as well. In this way the effects of cooperation between one or more members of an organization can be more finely evaluated and used to predict the success of different groups. In the football example there are various running backs, receivers, linemen, and one quarterback. The coach could see that if the quarterback had high performance indications for a pass play and that his two receivers are also at a high performance level, then a pass play would have greater chance for success than a mismatch of high and low performance indicators. Should the quarterback's predictive performance be low, then a running play with the quarterback handing off the ball may be safer. If the starting quarterback's predictive performance remains low, the coach may elect to put in the backup quarterback rather wait until a failure and pull the starting quarterback out after a bad play. Additionally, similar data can be collected on a single team's entire offense and defense, and then evaluated by scrimmaging against each other to compare various brain activity matchups and performance matchups.

Brain patterns may also be used to detect injury or other physical conditions of interest. For example, a quarterback may be sacked on a play and suffer a concussion. The quarterback may not notice the injury immediately or may not disclose the injury because he doesn't want to be held out of the game. However, if the subject's brain pattern was abnormal for the circumstance and did not match any other known pattern for the subject in his record, then the coach would have a strong indicator that a concussion may have occurred on the previous play and would be able to remove him from the game for an evaluation. In addition, EEG readings of subjects with concussions over time may be used to establish predictive patterns for the presence of a concussion.

As stated above, subject matter of this specification is not to be limited to sports applications. Alternative embodiments include applications where personal performance requires a high skill level and failure is possible. Examples are in military operations such as Special Forces raids or operations with little sleep or significant physical requirements. Persons with medical conditions could be automatically monitored if their condition was affecting their mental activity or mental performance. Managers of operation centers could monitor their employees to determine who needs a break or to shift persons around.

Embodiments also include applications where failure must be avoided. This embodiment would establish a requirement for a specific brainwave pattern to be present before an individual is allowed or permitted to perform a certain task. For example an airline pilot cannot consume alcohol for 8 hours prior to flying and must have 8 hours of sleep (“crew rest”) prior to flying a plane. Rather than simple rules, there are other factors that could impact a pilot's alertness or judgment such as family issues, worrying, medical, or mental issues. The subject matter of this specification would allow establishing a brainwave threshold by specific brainwaves of a composite activity level.

Another example would be doctors required to perform complex surgery. Physicians performing critical surgeries could be assessed in advance or in a near real time environment during surgery. Is the doctor's brainwave activity operating near his level where he has had success in the past or is it at a level where he has had problems? Another doctor could be used who is operating at a higher brainwave performance level and the first doctor may be relegated to simpler tasks.

A subject can also receive near real time feedback of his or her own performance levels. These “self-correcting” actions would alert a subject to get back up to normal mental performance, and then begin the critical tasks again. For example, a subject could detect their sleep deprivation levels and be alerted when a critical threshold is reached. The subject could then stop and rest for a short period of time. In another example, a subject could detect when their anger or anxiety levels are abnormal. The person could then talk to someone else on the issues that are distracting or impacting smooth performance.

The subject matter of this specification also contemplates the following embodiments:

A system for detecting a brain injury in an athlete, comprising a sensor device integrated into a helmet worn by an athlete that detects electrical activity taking place in the athlete's brain; a first communication device integrated into the helmet and operatively connected to the sensor device, that transmits a signal containing the electrical activity detected by the sensor device; a second communications device that receives the signal transmitted by the first communication device; and a computer, operatively connected to the second communication device and a display device, that displays the signal received by the second communication device.

A method for detecting a person's brain activity, comprising the steps of detecting electrical activity in a person's brain with electrical sensors; transmitting the detected electrical activity to a computer; comparing the detected electrical activity to patterns of electrical activity for the person's contained in a record stored in memory; determining a degree of similarity of each pattern of electrical activity in the record to the detected electrical activity; displaying the degree of similarity of each pattern of electrical activity on a display device; and predicting a person's performance level.

A method for detecting a group of person's brain activity, comprising the steps of detecting electrical activity in each person's brain with electrical sensors; transmitting the detected electrical activity to a computer; comparing the detected electrical activity to patterns of electrical activity for each person's contained in a record stored in memory; determining a degree of similarity of each pattern of electrical activity in the record to the detected electrical activity; displaying the degree of similarity of each pattern of electrical activity on a display device; predicting each person's performance level; assimilating each person's predicted performance in a group prediction model; and, predicting the group's performance in executing various group functions.

A method for detecting an athlete's brain activity, comprising the steps of detecting electrical activity in an athlete's brain with electrical sensors; transmitting the detected electrical activity to a computer; comparing the detected electrical activity to patterns of electrical activity for the athlete contained in a record stored in memory; determining a degree of similarity of each pattern of electrical activity in the record to the detected electrical activity; and displaying the degree of similarity of each pattern of electrical activity on a display device.

A system for detecting an athlete's mental status, comprising a sensor device that detects electrical activity that takes place in an athlete's brain; a first communication device, operatively connected to the sensor device, that transmits a signal containing the electrical activity detected by the sensor device; a second communications device that receives the signal transmitted by the first communication device; and a computer, operatively connected to the second communication device, that performs a method comprising the steps of comparing the detected electrical activity to patterns of electrical activity for the athlete contained in a record stored in memory; determining a degree of similarity of each pattern of electrical activity in the record to the detected electrical activity; and displaying the degree of similarity of each pattern of electrical activity on a display device.

A method for evaluating the probability of success of a sports tactic, comprising the steps of detecting electrical activity occurring in a brain of an athlete using a sensor device; wirelessly transmitting a signal that contains the detected electrical activity of the athlete using a first wireless communications device; wirelessly receiving the transmitted signal using a second wireless communication device; comparing the detected electrical activity to patterns of electrical activity for the athlete contained in a record stored in memory; determining a degree of similarity of each pattern of electrical activity in the record to the detected electrical activity; displaying the degree of similarity of each pattern of electrical activity on a display device; and selecting a sports tactic based on the degrees of similarity indicated on the display device.

A method for detecting brain injury in an athlete, comprising the steps of: detecting electrical activity occurring in a brain of an athlete using a sensor device; wirelessly transmitting a signal that contains the detected electrical activity of the athlete using a first wireless communications device; wirelessly receiving the transmitted signal using a second wireless communication device; comparing the electrical activity detected by the sensor device to patterns of electrical activity for the athlete stored in memory, which indicate brain injury; determining a degree of similarity of each pattern of electrical activity to the detected electrical activity; displaying the degree of similarity of each pattern of electrical activity on a display device.

A system for detecting a brain injury in an athlete, comprising a sensor device integrated into a helmet worn by an athlete that detects electrical activity taking place in the athlete's brain; a first communication device integrated into the helmet and operatively connected to the sensor device, that transmits a signal containing the electrical activity detected by the sensor device; a second communications device that receives the signal transmitted by the first communication device; and a computer, operatively connected to the second communication device and a display device, that displays the signal received by the second communication device.

A method for creating a record of patterns of electrical activity of an athlete under a set of desired circumstances; comprising the steps of monitoring the electrical activity occurring in the athlete's brain using a sensor device attached to the athlete's head while he is performing under desired circumstances; storing the monitored electrical activity in a record in memory along with desired information about concurrent circumstances; analyzing the stored electrical activity using pattern recognition software to determine if any patterns in electrical activity exist when the athlete is performing under specific circumstances; and storing information indicating what patterns arise under a given circumstance in the record containing that circumstance.

A system for detecting an person's mental status, comprising a sensor device that detects electrical activity that takes place in an person's brain; a first communication device, operatively connected to the sensor device, that transmits a signal containing the electrical activity detected by the sensor device; a second communications device that receives the signal transmitted by the first communication device; and a computer, operatively connected to the second communication device, that performs a method comprising the steps of comparing the detected electrical activity to patterns of electrical activity for the athlete contained in a record stored in memory; determining a degree of similarity of each pattern of electrical activity in the record to the detected electrical activity; and displaying the degree of similarity of each pattern of electrical activity on a display device.

A method for evaluating the probability of success of a performance tactic, comprising the steps of detecting electrical activity occurring in a brain of an athlete using a sensor device; wirelessly transmitting a signal that contains the detected electrical activity of the athlete using a first wireless communications device; wirelessly receiving the transmitted signal using a second wireless communication device; comparing the detected electrical activity to patterns of electrical activity for the athlete contained in a record stored in memory; determining a degree of similarity of each pattern of electrical activity in the record to the detected electrical activity; displaying the degree of similarity of each pattern of electrical activity on a display device; and selecting a sports tactic based on the degrees of similarity indicated on the display device.

A method for detecting brain injury in an individual, comprising the steps of: detecting electrical activity occurring in a brain of a person using a sensor device; wirelessly transmitting a signal that contains the detected electrical activity of the athlete using a first wireless communications device; wirelessly receiving the transmitted signal using a second wireless communication device; comparing the electrical activity detected by the sensor device to patterns of electrical activity for the athlete stored in memory, which indicate brain injury; determining a degree of similarity of each pattern of electrical activity to the detected electrical activity; and displaying the degree of similarity of each pattern of electrical activity on a display device.

A method for detecting brain injury in an athlete, comprising the steps of: detecting electrical activity occurring in a brain of an athlete using a sensor device; wirelessly transmitting a signal that contains the detected electrical activity of the athlete using a first wireless communications device; wirelessly receiving the transmitted signal using a second wireless communication device; comparing the electrical activity detected by the sensor device to patterns of electrical activity for the athlete stored in memory, which indicate brain injury; determining a degree of similarity of each pattern of electrical activity to the detected electrical activity; displaying the degree of similarity of each pattern of electrical activity on a display device.

A system for detecting a brain injury in an athlete, comprising a sensor device integrated into a helmet worn by an athlete that detects electrical activity taking place in the athlete's brain; a first communication device integrated into the helmet and operatively connected to the sensor device, that transmits a signal containing the electrical activity detected by the sensor device; a second communications device that receives the signal transmitted by the first communication device; and a computer, operatively connected to the second communication device and a display device, that displays the signal received by the second communication device.

A method for creating a record of patterns of electrical activity of an athlete under a set of desired circumstances; comprising the steps of monitoring the electrical activity occurring in the athlete's brain using a sensor device attached to the athlete's head while he is performing under desired circumstances; storing the monitored electrical activity in a record in memory along with desired information about concurrent circumstances; analyzing the stored electrical activity using pattern recognition software to determine if any patterns in electrical activity exist when the athlete is performing under specific circumstances; and storing information indicating what patterns arise under a given circumstance in the record containing that circumstance.

1. A system for detecting an person's mental status, comprising
a sensor device that detects electrical activity that takes place in an person's brain;
a first communication device, operatively connected to the sensor device, that transmits a signal containing the electrical activity detected by the sensor device;
a second communications device that receives the signal transmitted by the first communication device; and
a computer, operatively connected to the second communication device, that performs a method comprising the steps of
comparing the detected electrical activity to patterns of electrical activity for the person contained in a record stored in memory;
determining a degree of similarity of each pattern of electrical activity in the record to the detected electrical activity; and
displaying the degree of similarity of each pattern of electrical activity on a display device.

2. The system of claim 1, wherein the sensor device detects total power of an EEG of the person's brain and the patterns of electrical activity for the person contained in the record are patterns of total power of the EEG of the person's brain.

3. The system of claim 1, wherein the sensor device is integrated into a helmet worn by the person and the person is an athlete.

4. The system of claim 1, wherein the sensor device is integrated into a helmet worn by the person and the person is an athlete, the sensor device detects total power of an EEG of the person's brain, and the patterns of electrical activity for the person contained in the record are patterns of total power of the EEG of the person's brain.

5. A system for detecting a brain injury in an athlete, comprising
a sensor device integrated into a helmet worn by an athlete that detects electrical activity taking place in the athlete's brain;
a first communication device integrated into the helmet and operatively connected to the sensor device that transmits a signal containing the electrical activity detected by the sensor device;
a second communications device that receives the signal transmitted by the first communication device; and
a computer, operatively connected to the second communication device and a display device, that displays the signal received by the second communication device.

6. A method for evaluating the probability of success of a performance tactic, comprising the steps of
detecting electrical activity occurring in a brain of a person using a sensor device;
wirelessly transmitting a signal that contains the detected electrical activity of the person using a first wireless communications device;
wirelessly receiving the transmitted signal using a second wireless communication device;
comparing the detected electrical activity to patterns of electrical activity for the person contained in a record stored in memory of a computer;
determining a degree of similarity of each pattern of electrical activity in the record to the detected electrical activity;
displaying the degree of similarity of each pattern of electrical activity on a display device. and
selecting a tactic based on the degrees of similarity indicated on the display device.
